SEQUENCE LISTING (12 sequences)

SEQ ID NO: 1 - protein, 551 aa, Homo sapiens
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVKKRDLEQAISQRIQTNNNPFQVPIEEQRGDYDLNAVRLCFQVTVRDPSGRPLRLPPVLPHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 2 - protein, 551 aa, Homo sapiens; identical to SEQ ID NO: 1

SEQ ID NO: 3 - protein, 558 aa, Gallus gallus
MEPADLLPLYLQPEWGEQEPGGATPFVEILEQPKQRGMRFRYKCEGRSAGSIPGEHSTDSARTHPTIRVNHYRGPGRVRVSLVTKDPPHGPHPHELVGRHCQHGYYEAELSPERCVHSFQNLGIQCVKKRELEAAVAERIRTNNNPFNVPMEERGAEYDLSAVRLCFQVWVNGPGGLCPLPPVLSQPIYDNRAPSTAELRILPGDRNSGSCQGGDEIFLLCDKVQKEDIEVRFWAEGWEAKGSFAAADVHRQVAIVFRTPPFRERSLRHPVTVRMELQRPSDRQRSPPLDFRYLPHQGDLQCIEEKRKRTRDTFRAFVQRAPLPGLEPNPEPRPPRRIAVPSRPPPAPQQPPSMVGAPPAPLFPLGVPPASSPTPEPLAEALLQLQFDDGVGGSGPPPSTTTTTTTTQCALGGGIPDPGGSPLDLGALLGDPPFDTIDAAELQRLLGPPETPPGGIGAGGGFGELLSLPTNFGDPPSSTAATFGPSPPMLLSYPEAITRLVQCQTPGGSGGGGPPVGPPQDLGGPLHPPGAPPQPTEDSLPSLGDLDFSAFLSQFPSS

SEQ ID NO: 4 - protein, 558 aa, Gallus gallus; identical to SEQ ID NO: 3

SEQ ID NO: 5 - protein, 551 aa, Homo sapiens; identical to SEQ ID NO: 1

SEQ ID NO: 6 - protein, 537 aa, Homo sapiens
MDELFPLIFPAEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVKKRDLEQAISQRIQTNNNPFQVPIEEQRGDYDLNAVRLCFQVTVRDPSGRPLRLPPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTAQRPPDPAPAPLGAPGLPNGLLSGDEDFSSIADMDFSALLSQISS

SEQ ID NO: 7 - protein, 537 aa, Homo sapiens; identical to SEQ ID NO: 6

SEQ ID NO: 8 - protein, 550 aa, Homo sapiens [sequence omitted]

SEQ ID NO: 9 - protein, 550 aa, Homo sapiens [sequence omitted]

SEQ ID NO: 10 - DNA, 21 bp, Artificial
  primer to amplify p65 cDNA
ggccatggacgaactgttccc

SEQ ID NO: 11 - DNA, 20 bp, Artificial
  primer to amplify p65 cDNA
ggagggtccttggtgaccag

SEQ ID NO: 12 - protein, 7 aa, Artificial
  fluorogenic substrate to measure caspase activity
XEVDAPX

(19) United States
(12) Patent Application Publication
(10) Pub. No.: US 2005/0287158 A1
(43) Pub. Date: Dec. 29, 2005

(54) The p65 subunit of nf-kb for the radiosensitization of cells

(75) Inventors: Mira Jung, Washington, DC (US); Anatoly Dritschilo, Washington, DC (US)

Correspondence Address: PILLSBURY WINTHROP SHAW PITTMAN, LLP, P.O. BOX 10500, MCLEAN, VA, 22102, US

(73) Assignee: Georgetown University, Washington, DC (US)

(21) Appl. No.: 10/504,364

(22) PCT Filed: Feb. 19, 2003
(86) PCT No.: PCT/US03/04513

§ 371 Date: Jul. 13, 2005

Related U.S. Application Data
(60) Provisional application No. 60/356,748, filed on Feb. 15, 2002.

(51) Int. Cl. A61K 39/00; A61K 38/18

(52) U.S. Cl. 424/185.1; 514/12

(57) ABSTRACT

The transcription factor NF-κB is activated in response to various stimuli including ionizing radiation. Disruption of NFκB activation by mutant forms of the NF-κB inhibitor IκB-α or by proteasome inhibitors enhances both sensitivity to radiation and radiation-induced apoptasis. The present invention shows that expression of a dominant negative fragment of human p65 (p65DN) leads to down-regulation of both endogenous p65 protein and its mRNA. The dominant negative protein also inhibits radiation-induced NF-κB activation by preventing the proteolysis of IκB-α, resulting in enhancement of cellular radiosensitivity and radiation-induced apoptosis. The region of p65 in the dominant negative fragment is thus a molecular target for disruption of NF-κB activation and sensitization of tumors to radiotherapy.

### RELATED APPLICATION

This application relates to U.S. Provisional Ser. No. 60/356,748, filed on Feb. 15, 2002, and which is incorporated in its entirety by reference herein.

### FIELD OF INVENTION

This invention relates to methods for inhibiting or reducing cancerous growth, sensitivity to cytotoxic agents and immune inflammation. In particular, the methods involve the inhibition or reduction of NF-κB activation in cancer or inflammatory cells with the use of dominant negative DNA and protein constructs. Such methods find utility in disease treatment regimens, particularly treatment of cancers.

### BACKGROUND OF INVENTION

The NF-κB nuclear transcription factor plays a role in cell differentiation, immune response, cell proliferation and cell death (1-3). Expression of multiple genes implicated in inflammation, including proinflammatory cytokines and their receptors, is under the transcriptional control of NF-κB. Exposure of cells to ionizing radiation induces NF-κB activation, and the transcriptional activation of cell survival genes (10-13). A recent study has demonstrated that a novel radiation-induced signaling pathway for NF-κB activation includes ATM, a gene that is mutated in AT patients, rendering such patients extremely sensitive to radiation (13). Therefore, inhibition or reduction of NF-kB-mediated gene expression increases the radiosensitivity of cells.

The NF-κB family consists of five Rel members, including NF-κB1 (p50 and p105), NF-κB 2 (p52 and p100), c-Rel, RelA (p65), and RelB (4). NF-κB forms homo- or heterodimers of Rel proteins that share a Rel homologous domain at the NH₂-terminus, including a conserved DNA binding and dimerization domain (5). For instance, RelA (p65) contains a conserved DNA binding region in the NH₂ terminal region comprising amino acids 1-300, and a transcriptional activation domain in the carboxy terminal region, with a nuclear localization signal at residues 301 to 304 (4-6).

In resting cells, NF-κB is bound to the inhibitory IκBproteins (6). Some IκBproteins accomplish inhibition by sequestering NF-kB p65 in the cytoplasm and inhibiting nuclear import (IκB-βand IκB-ε), while others, i.e., IκB-ε accomplish inhibition by shifting the balance between nuclear import and export of Rel proteins (48). Exposure of cells to TNF-α, phorbol esters or DNA damaging agents, results in IκB-phosphorylation, ubiquitination, and degradation, leading to NF-κB nuclear translocation and transcription activation NF-κB-regulated genes (7, 8).

The mechanism of NF-κB activation involving signaling-induced phosphorylation on Ser 32 and 36 by a complex of three polypeptides (the IKK complex) and subsequent degradation by proteosomes has well been characterized (9, 49, 50). A further explanation recently advanced is that phosphorylation of IκB promotes its association with the Src homology 2 domains of p85, one of the P13 kinase regulatory subunits, thereby displacing NF-κB which is then translocated to the nucleus (47). Nevertheless, whether by degradation or via p85 binding of IκB, displacement of NF-κB from IκB is thought to lead to its nuclear translocation by virtue of exposure of the nuclear localization signal domain (4-6).

Since NF-κB activation may impact on cell cycle, apoptosis and other functions critical for cellular survival or death, we and others have investigated the potential for radiation sensitization of cancer cells by interruption of NF-κB signaling (14-16). Such direct strategies have demonstrated enhancement of cells undergoing apoptosis, but the effect on cellular radiosensitization has been difficult to ascertain (17, 18). Most studies have used vector-mediated expression of mutant IκB-α or chemical proteosome inhibitors, which interfere with IκB-α degradation, to regulate intrinsic NF-κB activation following exposure to genotoxic stresses (19-24). Although such small molecule inhibitors enhanced antitumor effects in in vivo xenografts, including human breast, head and neck, pancreatic, and prostate (25-29), criticisms of such results center on non-specific effects of the tested chemicals.

To test the effects of directly modulating NF-κB activation in human head and neck cancer cells, we targeted NF-κB p65 subunit expression by expressing a dominant negative p65 construct. The dominant negative (DN) p65 construct tested contains the initial 250 bp of the Rel homologous DNA binding domain, but lacks the nuclear localization signal and transcription activation domain. We evaluated the effects of NF-κB p65 (DN) expression on squamous carcinoma cells by measuring radiosensitivity and apoptosis following ionizing radiation exposure. It was surprisingly found that expression of the dominant negative construct resulted in a down-regulation of both NF-κB activation and p65 gene expression, apparently by competing for IκB binding in the cytoplasm, thereby resulting in radiosensitivity and radiation-induced apoptosis of the carcinoma cells.

### SUMMARY OF THE INVENTION

The present invention relates to methods of inhibiting or reducing NF-κB activation in cells by expressing a dominant negative (DN) NF-κB subunit or fragment thereof in the cells. The invention particularly involves exposing cells to a DN derivative of the p65 (ReIA) subunit, which contains a fragment derived from the amino terminal portion of the native protein and which binds to the IκB-α inhibitory protein. Exposure of cells to ionizing radiation normally activates NF-κB, leading to the expression of cell survival genes regulated by activated NF-κB. Therefore, exposure of cells to inhibitors of NF-κB activation enhances sensitivity of cells to ionizing radiation and increases radiation-induced apoptosis. Such methods are particularly useful for increasing the radiosensititvity of tumor cells during cancer treatments utilizing radiation-based therapies.

As mentioned briefly above, it was surprisingly found that exposure of cells to a DN p65 construct resulted in a down-regulation of NF-κB activation, apparently by competing for IκB binding in the cytoplasm, thereby resulting in radiosensitivity and radiation-induced apoptosis of the carcinoma cells. This was truly surprising when taken in view of the accepted model of NF-κB activation known at the time, which describes the disassociation of NF-κB from IκB leading to NF-κB activation and up-regulation of NF-κB-mediated gene expression. This model is not entirely consistent with the surprising discovery of the present invention, because the model would predict that competition for IκB binding would lead to an increase in disassociated NF-κB and a higher level of NF-κB-mediated gene expression. This suggests that it is more than the mere release of NF-κB from IκB that promotes its activation and translocation, but that some type of modification to NF-κB occurs in the release process that enables NF-κB activation.

Thus, it would be counterintuitive to predict that a competing NF-κB subunit or fragment would mediate decreased NF-κB activation in view of the increase in free NF-κB as a result of such competition. In addition to causing a decrease in NF-κB signal-induced activation, expression of the dominant negative construct in cells also led to a decrease in p65 gene expression, possibly through a negative feedback mechanism resulting from an excess of non-activated NF-κB. Given that exposure of cells to the DN construct results in a decrease of both NF-κB activation and p65 gene expression, pharmaceutical products based on the DN construct provide a powerful means to regulate NF-κB-mediated gene expression.

### BRIEF DESCRIPTION OF THE DRAWINGS

FIG. 1. Effects of DN p65 on p65 transcript and protein expression. Total RNAs were isolated from SCC-35 cells and clonal cells expressing DN p65 and analyzed by Northern analysis (NA). A) The expression of the NH₂-terminus p65 (DN) was detected by Northern analysis. The endogenous p65 RNA expression level in DN p65 clonal cells was compared to the controls (SCC-35) (arrowhead). The arrow indicates the message for the NH₂-terminus transcript. B) Total cell extract was analyzed for the p65 protein expression by Western blotting in representative clonal cells expressing DN/CL6 p65.

FIG. 2. Effects of DN p65 on (ionizing radiation). IR-induced NF-κB activation. A) Nuclear p65 and phosphorylation of Ser 32 IkB-α in the cytoplasmic fraction of cells were determined at indicated time intervals following irradiation by Western analysis using antibodies to p65 and Ser 32 IκB-α. The quantity control of samples was determined by probing the same membrane with anti-Actin antibody. B) Cells were transfected with the NF-κB luciferase reporter construct. After transfection and irradiation with 20 Gy of IR or treatment with TNF-□ (10 ng/ml), cell extracts were prepared at 6 hr and used for luciferase assays. Values were represented as the relative luciferase activities for irradiated or treated cell extract to that for the control.

FIG. 3. Radiation survival assays. Logarithmically growing cells were exposed to graded gamma radiation doses. Clonogenic survival was determined by counting colonies containing >50 cells after two weeks of growth. A semi-logarithmic plot of the data fore these cells is shown. Points and bars mean +/−SEM from triplicate flasks in each experiment.

FIG. 4. Effects of IR on the apoptosis. (A) Effects of various doses of IR on the apoptotic index and (B) kinetics of IR-induced apoptotic index at various intervals. Cells were exposed to 2-20 Gy of IR. At the indicated times thereafter (in all experiments, time zero refers to cells that were subjected to sham irradiation), attached and floating cells were collected and the number of apoptotic cells was determined as a percentage of the total number of cells (apoptotic index). Data are means±SD of values from three independent experiments. (C) PARP cleavage was measured after radiated intervals. Cell lysates were subjected to immunoblot analysis with antibodies to PARP. The arrowhead and arrow indicate uncleaved and cleaved products of PARP, respectively. (D) Effects of IR on caspase-3 activity. Cells were exposed to 5 Gy of IR, and attached and floating cells were collected at the indicated times thereafter. Cells were untreated or treated with 50 μM Ac-DEVD-CHO (caspase-3 inhibitor) 2 hr prior to IR exposure. Cell lysates were prepared and assayed for caspase-3 activity with a specific fluorogenic peptide substrate. The caspase activities were determined at indicated intervals. Data in (C) are representative of three independent experiments; those in (D) are means±SD of values from three independent experiments.

FIG. 5. This figure contains the amino acid sequences of various NF-κB subunits. Including human NF-κB p65 subunit and splice variants thereof.

### DETAILED DESCRIPTION OF THE INVENTION

The present invention encompasses methods of inhibiting or reducing NF-κB activation in a cell comprising exposing the cell to a dominant negative (DN) NF-κB subunit or fragment thereof such that NF-κB activation is inhibited. Preferably, the DN NF-κB subunit or fragment thereof is a dominant negative p65 subunit or p65 fragment, which preferably comprises all or part of the DNA binding domain of p65. For instance, a DN p65 fragment according to the invention may comprise amino acids ranging from about 1 to about 200, or to about 250, or to about 300 of native p65.

Preferably, a DN p65 fragment according to the invention does not contain an active nuclear localization sequence, which is located at amino acid residues 301-304 of human p65, nor an active transcriptional activation domain, which is located at the carboxy terminus. However, it should be understood that DN variants fused to amino acids taken from these regions but not sufficient to denote the functions associated with these regions would also be encompassed by the present invention.

Preferably the DN p65 subunit or p65 fragment of the invention comprises an IκB-alpha binding domain. Such a DN construct may comprise amino acids 1-101 of native p65, or any of the amino acids from native p65 ranging from about 1 to about 100. Most preferably, the DN p65 subunit or p65 fragment according to the invention comprises amino acids from native p65 ranging from about 1 to about 85, with a particularly preferred construct consisting of amino acids 1-84 from native p65.

The present invention encompasses polynucleotides, e.g., DNAs, cDNAs, mRNAs, gene expression constructs, vectors, etc. that encode and drive the expression of the DN peptides of the present invention. Polynucleotides that are degenerate variants, or polynucleotides that encode biologically active variants or fragments or spliced derivatives of the DN subunits and fragments disclosed herein are also included in the invention. Promoters for driving the expression of the DN polynucleotide in any given cell may be gleaned from the art, and may be engineered to drive the expression of a DN polynucleotide using routine molecular cloning techniques. In this regard, the three volumes of Molecular Cloning, A Laboratory Manual, 2^{nd} ed., by Sambrook et al. (1989) are herein incorporated by reference.

Polypeptide variants are also encompassed, which include peptides that are altered by one or more amino acids but which retain the inhibitory function of the disclosed DN peptides. Such a variant can have “conservative” changes, wherein a substituted amino acid has similar structural or chemical properties, e.g., replacement of leucine with isoleucine. More rarely, a variant can have “nonconservative” changes, e.g., replacement of a glycine with a tryptophan. Similar minor variations can also include amino acid deletions or insertions, or both. Guidance in determining which and how many amino acid residues may be substituted, inserted or deleted without abolishing biological activity can be found using computer programs well known in the art, for example, DNAStar software.

The methods of the invention are accomplished by exposing a target cell to a DN construct either in vitro or in vivo, wherein exposure may be mediated by transfecting the cell with a polynucleotide encoding the DN NF-κB subunit or fragment thereof, and expressing the dominant negative NF-κB subunit or fragment thereof encoded by the polynucleotide such that NF-κB activation is inhibited or reduced. Exposure may also be mediated by exposing a cell to a DN peptide directly, for instance by contacting the cell with a DN NF-κB subunit or fragment thereof coupled to an internalization moiety. Suitable internalization moieties are known in the art, and for instance may be selected from the group consisting of a peptide internalization sequence, a liposome, and an antibody or an antibody fragment or ligand that binds to a surface receptor on the target cell. Such internalization moieties are described in U.S. Pat. No. 6,303,576, which is herein incorporated by reference in its entirety.

The methods of the invention result in the modulation of expression of at least one NF-κB-regulated gene. For genes positively regulated by NF-κB, the methods of the invention will result in down-regulation of gene expression. In contrast, for genes negatively regulated by NF-κB, for instance because NF-κB is involved in activating the transcription of a negative regulatory protein, the methods of the invention may result in the up-regulation of gene expression. In particular, the methods of the invention result in a down-regulation of gene expression of the gene encoding native p65.

The methods of the invention also result in the phosphorylation of IκB proteins, but not their degradation. In particular, IκB-alpha is phosphorylated but not degraded by the methods disclosed herein. Without wishing to be bound to any particular theory, the applicants believe that the DN peptides according to the invention compete with native NF-κB for IκB-alpha binding, and sequester IκB following phosphorylation such that it cannot be degraded. It is quite surprising that such a competition would be accompanied by a down-regulation of native NF-κB activation, given that sequestering of IκB would mean that there is more free NF-κB to be translocated into the nucleus. This suggests that something more is required than the mere disassociation from IκB, further suggesting that some change occurs in NF-κB via its association with IκB that enables its subsequent activation, such as, for instance, a change in conformation or a physical or chemical modification.

The methods of the invention may be used to increase the sensitivity of a target cell to ionizing radiation. As a result of NF-κB down-regulation and the lack of expression of radiation-induced cell survival genes, a target cell undergoes radiation-induced apoptosis at an increased rate over a cell that has not been exposed to a DN NF-κB peptide. This attribute of the methodology disclosed herein renders the methods particularly useful for the targeting of tumor cells, to increase the efficacy of radiation-based therapies.

The invention also encompasses compositions and particularly therapeutic compositions comprising the DN polynucleotides and peptides described herein. In vivo delivery of polynucleotide compositions may be mediated by any known delivery method, i.e. retroviral delivery or liposomal delivery. A preferred method is described in U.S. Pat. No. 6,333,396, which discloses an effective means of delivering a nucleic acid to a target cell by complexing the nucleic acid with a single chain antibody specific for a receptor on the surface of the target cell. For cancer cells, receptors commonly over-expressed in such cells may be targeted, i.e., the EGF receptors. Other examples of cell specific targeting include ex vivo gene transfer to specific cell populations such as lymphocytes and direct injection of DNA into muscle tissue or other organs or cell masses as described in U.S. Pat. No. 6,214,804, herein incorporated by reference in its entirety. The peptides of the invention may also be targeted to specific cells in vivo using liposomes or antibodies or other techniques known in the art, i.e., using the internalization moieties described herein.

Once formulated, the therapeutic compositions of the invention may be conventionally administered parenterally, e.g., by injection, either subcutaneously or intramuscularly. Additional formulations suitable for other modes of administration include oral and pulmonary formulations, suppositories, and transdermal applications depending on the target cell and the mode of delivery. Dosage treatment may be a single dose schedule or a multiple dose schedule, administered in conjunction with, either sequentially or simultaneously, with other therapeutic agents. Such agents include antisense and other nucleotide constructs derived from the coding sequence of p65, such as polynucleotides that mediate RNA interference. Of particular interest are antisense polynucleotides capable of inhibiting transcription and/or translation of a NF-κB subunit gene either in vitro or in vivo.

With regard to cancer therapies, the disclosed methods may be employed in conjunction with chemotherapeutic or radiation-based treatments, with radiation-based treatments being particularly preferred. Since NF-κB is involved in the expression of cell survival genes in general, the DN constructs of the invention find use in combined therapeutic regimens outside of those that include radiation exposure. Suitable cancers to be treated by the methods disclosed herein include breast, colon, throat, lung, skin, pancreatic, ovarian, etc. as well as blood-based cancers such as lymphoma and leukemia.

The methods of the present invention also find use in the treatment of inflammatory conditions. NF-κB is involved in activating gene expression in immune cells involved in inflammatory responses. Therefore, the methods and compositions of the present invention may also be used to treat immune-related disorders. Such methods may involve direct injection of either the polynucleotide or peptide DN constructs of the invention at the site of inflammation.

Suitable disease targets include any disorder associated with inflammation, such as inflammatory diseases selected from the group consisting of arthritis, ataxia-telangiectasia, asthma, inflammatory bowel disease, etc.

It should be appreciated that the DN polynucleotides and peptides disclosed herein find other uses than in in vivo therapy. For instance, because the DN constructs described herein provide a more direct route of down-regulating NF-κB activation than other methods of the prior art, the DN polynucleotides and peptides of the invention may be administered to cells in vitro in order to determine whether a given gene is subject to NF-κB regulation. The DN constructs may also be administered in an in vitro setting in order to inhibit or reduce NF-κB-mediated gene expression to more clearly focus on inter-related genetic regulatory pathways.

The amino acid and DNA sequences for NF-κB subunits, in particular the p65 subunit and splice variants thereof are well known in the art.

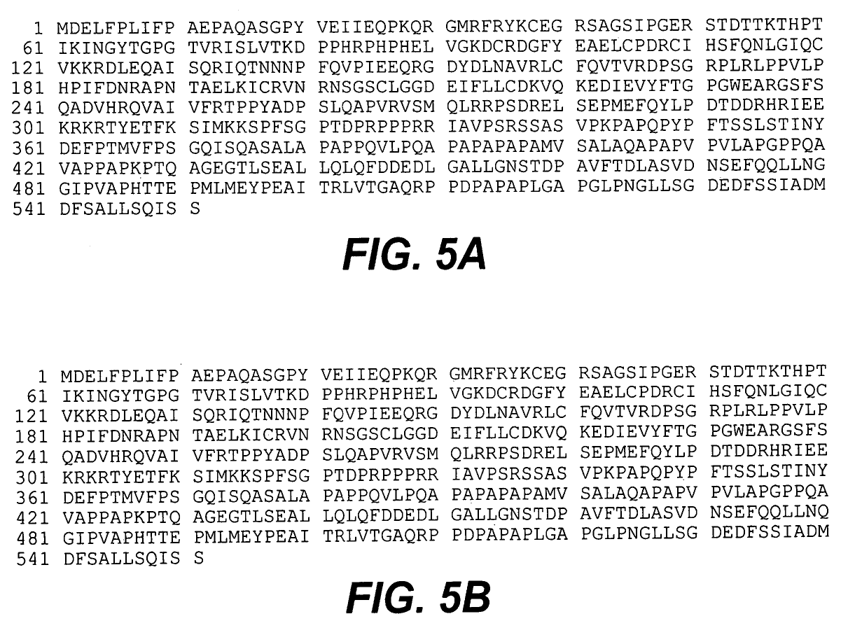

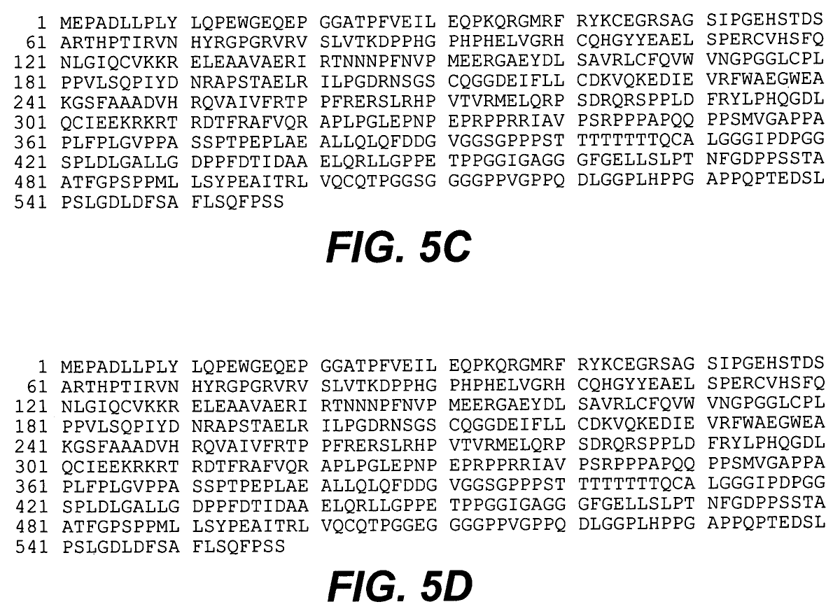

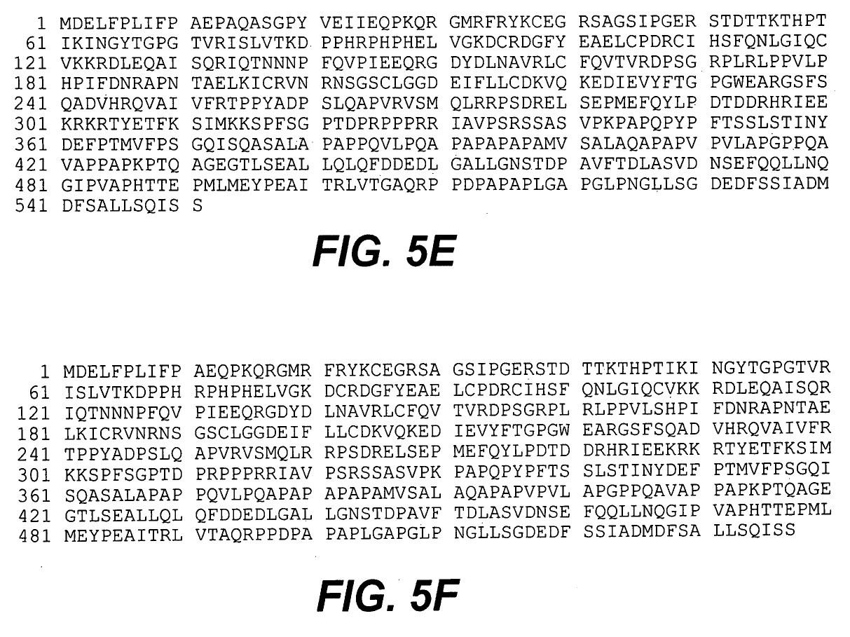

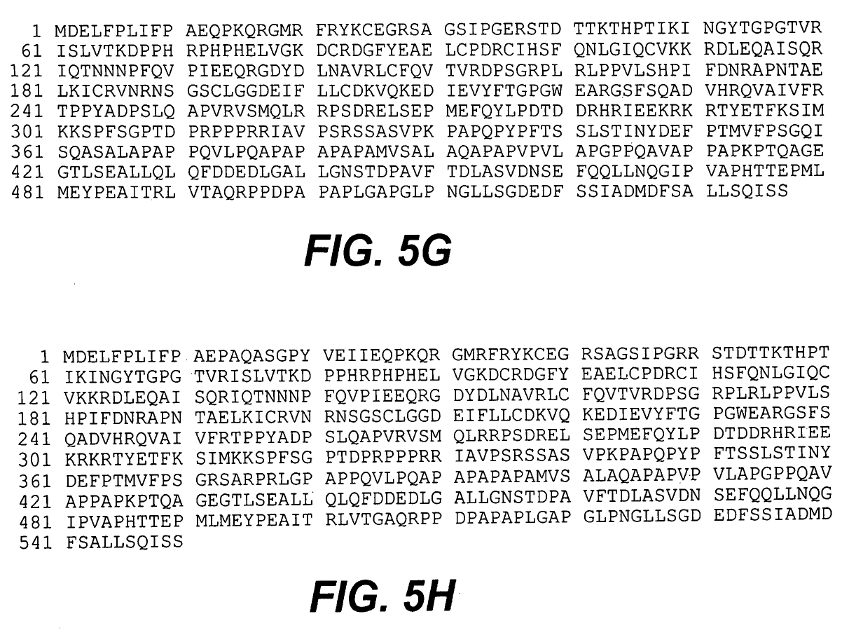

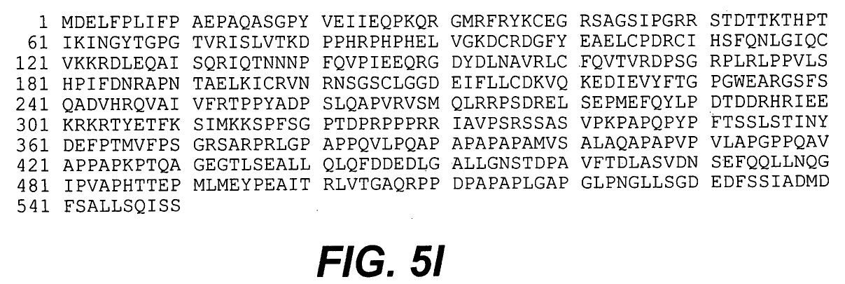

The amino acid sequences of different NF-kappa B p65 subunits and splice variants are contained in FIG. 5 of this application.

The invention embraces the use of any DNA encoding a dominant negative NF-κB subunit or fragment or splice variant or mutation thereof that results in an inhibition or reduction of NF-κB activation in a cell, e.g., a cancer cell. Preferably, the subunit, fragment, variant or mutant will comprise the amino acids of native p65 ranging from at least about 1-84.

The following further examples describe, without limiting, the nature of the invention.

### EXAMPLES

Materials and Methods

Cell culture, Transfection and Clonal Selections: Human head and neck squamous carcinoma cells (SCC-35) were used for the experiments described. These cells have been previously characterized as resistant to ionizing radiation based on clinical and radiobiological parameters (30). Cells were cultured in 10 cm² dishes and maintained in Dulbeco modified Eagle's medium supplemented with 10% fetal bovine serum, 2 mML-amino acids. Cells were passaged routinely at 80-90% confluence and checked for mycoplasma contamination at three-month intervals. For transfection and clonal selections, logarithmically growing cells were transfected with 5 μg of plasmid DNA, using Lipofectamine (5 μg/ml) (BRL) as suggested by the manufacturer (GIBCO). For the stable clonal selection, cells were transfected with the plasmid DNA containing the NH2-terminus p65 by using Lipofectamine (Gibco) as suggested by the manufacturer. G418 resistant clonal cells were then subcloned and maintained in the complete medium containing G418 (400 μg/ml) as described above. Drug resistant colonies were pooled or subcloned for future studies.

Construction of the NH₂-terminus (a.a. 1-84) p65: The NH₂-terminal 250 bp fragment of p65 cDNA was prepared by performing PCR, using the full-length human cDNA as a template (31). The primers, H65F (ggccatggacgaactgttccc) (SEQ ID NO: 1) and H65R (ggagggtccftggtgaccag) (SEQ ID NO: 2), were used to amplify the target fragment. The PCR product was subcloned into plasmid pCR3.1 (Invitrogen), which confers G418 resistance and an expression in eukaryotic cells. The inserts were confirmed by automated sequence analysis. Plasmid DNA was prepared by using CsCl-ethidium bromide density gradient centrifugation.

RNA analysis: Total RNA was isolated from cells using the RNAzol B as suggested by the manufacturer (Tel-Test Inc.) and subjected to Northern analysis. RNA was electrophoresed on a 1% formaldehyde-agarose gel and then transferred to the nitrocellulose membrane, followed by UV cross-linking. The membrane was hybridized with γ-³²P-dATP labeled cDNA probes for 16 h, washed, and autoradiographed.

Western blotting: Following exposure to γ-radiation (1-20 Gy) (a ¹³⁷Cs J. L. Shepherrd Mark 1 laboratory irradiator) or TNF-α (10 ng/ml) treatment, cell extracts were prepared by using lysis buffer (50 mM Tris-Cl, pH 7.5, 100 mM NaCl, 0.1% NP40, 1 mM PMSF, 10 mg/ml leupeptin, 1 mg/ml aprotinin, 1 mg/ml perpstatin), followed by centrifugation at 12,000×g for 20 minutes. Protein quantities were determined using the BCA protein assay kit (Pierce). Equal amounts of cell extracts were analyzed on 10% SDS-polyacrylamide gels. The transblotted membranes were blocked in PBS containing 5% dry milk for 1 h and probed with specific antibodies, followed by washing with PBST. The membrane was then incubated with horse-radish peroxidase conjugated secondary antibodies for 30-60 minutes washed with PBST, and developed by using the ECL-chemiluminescence detection system (Amersham). Equal loading and transfer of proteins among lanes were confirmed by immunoblot analysis with antibodies to β-actin (Amersham) or by staining the membrane with Ponceau S. Other antibodies used in this study include anti-NF-κB(p65), IκB-α caspases-3, (Santa Cruz Biotechnology, Santa Cruz, Calif.) and PARP (Enzyme Synthesis, Dublin, Calif.).

Luciferase Reporter Assay: Cells were transiently transfected for 16 h with 5 μg/ml of plasmid DNA by using Lipofectin (Gibco-BRL). Following treatment with ionizing radiation (20 Gy) or TNF-α (10 ng/ml), cells were lysed at 6 h in Reporter Lysis Buffer (Promega), and centrifuged for 2 min at 14,000×g. The cell extracts were then used to measure β-galactosidase and luciferase activities as suggested by the manufacturer (Promega).

Radiation survival assay: Clonogenic dose response was determined as previously described (32). Briefly, exponentially growing cells were plated in T-25 flasks and exposed to graded doses of ionizing radiation in a ¹³⁷Cs J. L. Shepherrd Mark 1 laboratory irradiator with a dose accuracy of ±5%. Physics support for calibration and quality assurance was provided for all experimental protocols. Colony counts were fit to single hit multitarget (SHMT) and the linear quadratic (L-Q) models (33). Data points were obtained from two to three independent experiments in triplicates for each experiment.

Apoptotic Index: Adherent and floating cells were collected at various times after exposure to various doses (1-20 Gy) of gamma radiation (a ¹³⁷Cs J. L. Shepherd Mark 1laboratory irradiator) and stained with acridine-orange and ethidium bromide (14). About 600 cells (˜100 cells per field) were scored for the aberrant chromosome organizations under flourescence microscopy. The apoptotic cells=(total number of cells with apoptotic nuclei/total number of cells counted)×100. All values represent the mean±SE of the experiments. All experiments were performed more than three times.

Measurement of caspase activities—The activity of caspases-3 was assayed fluorometrically with specific fluorogenic substrates: MOCAc-Asp-Glu-Val-Asp-Ala-Pro-Lys(DNP)-NH₂ (Peptide Institute, Osaka, Japan) (34). Cell extracts (60 μg of protein) were incubated for 1 h at 37° C. with 600 nM substrate in a reaction mixture (500 μl) containing 10 mM Hepes-NaOH (pH 7.4), 40 mM P-glycerophosphate, 50 mM NaCl, 2 mM MgCl₂, 5 mM EGTA, 1 mM DTT, 2 mM ATP, 10 mM creatine phosphate, and creatine kinase (50 μg/ml). The fluorescence of the cleaved substrates was measured with a Hitachi F-4500 spectrophotometer at excitation and emission wavelengths, respectively, of 328 and 393 nm for MOCAc-DEVDAPK(DNP)-NH₂.

Effects of p65DN on the Expression of Endogenous p65 and the Activation of NF-κB

The NF-κB heterodimer composed of p50 and p65 subunits is abundant in most cell types. The p65 subunit contains a DNA-binding domain in its NH₂-terminal region (residues 1 to 300), a nuclear localization signal in its central region (residues 301 to 304), and a transcriptional activation domain in its COOH-terminal region (4, 5). Deletion of the DNA-binding domain of p65 has previously been shown to interfere with NF-κB function (35).

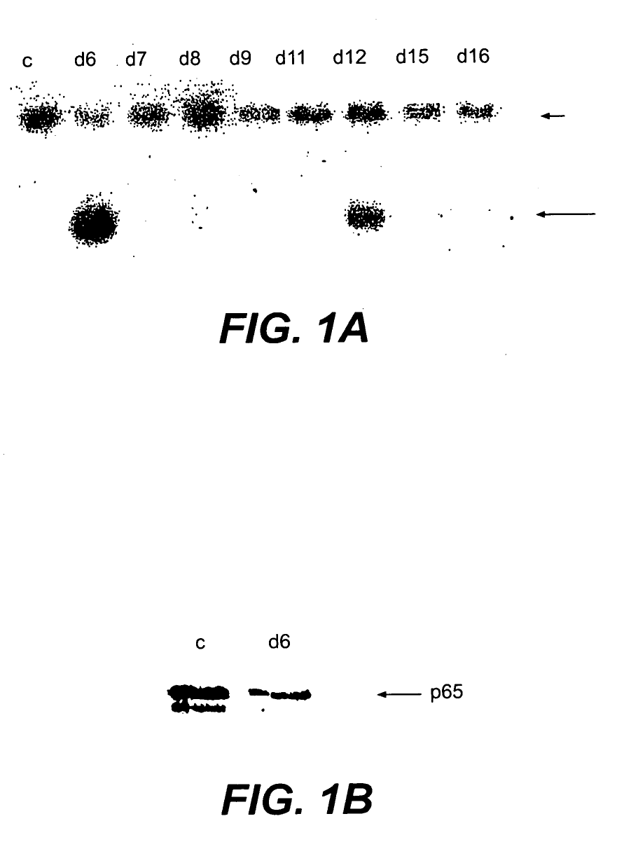

To investigate the effects of targeting p65 as a means of disrupting NF-κB activation, we transfected radioresistant human squamous carcinoma SCC-35 cells with an expression vector encoding amino acids 1 to 84 of human p65 (p65DN). The resulting G418-resistant colonies were isolated and analyzed for expression of p65DN and endogenous p65. Northern blot analysis revealed that the cell clones contained both endogenous p65 mRNA and transcripts corresponding to p65DN (FIG. 1A); however, the amount of p65 mRNA was reduced compared with that in the parental SCC-35 cells. Of these clonal cells, the line DN/CL6, which contained the largest amount of p65DN mRNA, was characterized further. Immunoblot analysis with antibodies to p65 revealed that the amount of endogenous p65 protein in DN/CL6 cells was reduced by 75% compared with that in the parental SCC-35 cells (FIG. 1B). These data demonstrate that expression of p65DN resulted in a marked reduction in the amounts of endogenous p65 mRNA and protein, suggesting that the mutant functions as a dominant negative regulator of NF-κB function.

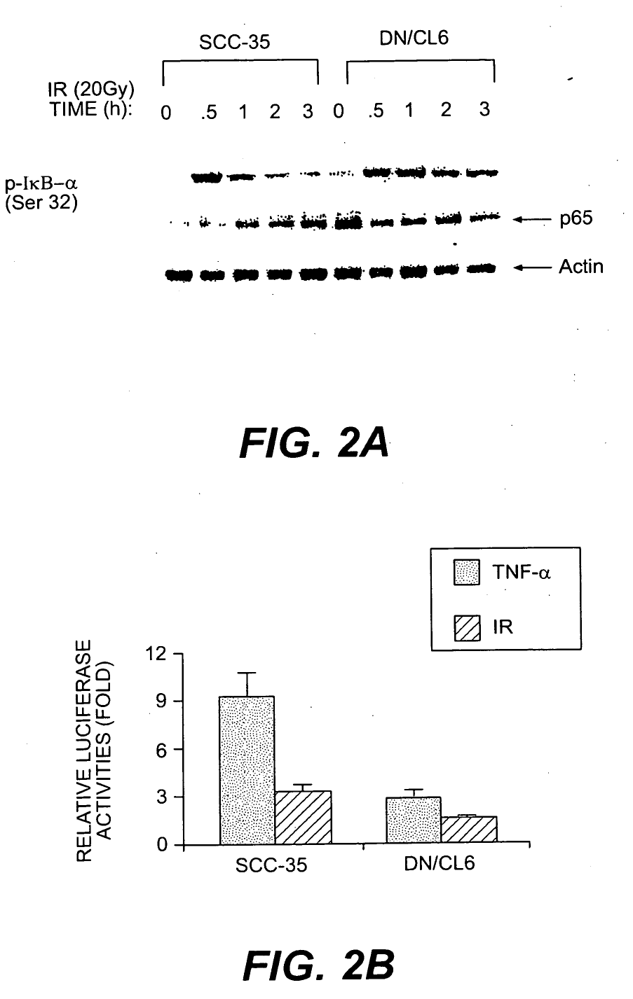

IκB-α binds to both subunits of the p50-p65 heterodimer. We therefore next determined the effect of p65DN expression on IR-induced phosphorylation and subsequent degradation of IκB-αin the cytoplasm. Immunoblot analysis with antibodies specific for IκB-α phosphorylated on Ser³² revealed that phosphorylation of IκB-α on this residue was apparent within 30 min of irradiation of SCC-35 cells and thereafter decreased (FIG. 2A). However, in DN/CL6 cells, IR induced an increase in the phosphorylation of IκB-A that was also apparent at 30 min but which remained markedly increased at 3 h. Furthermore, whereas IR induced a gradual increase in the amount of p65 in the nucleus of SCC-35 cells, irradiation resulted in a decrease in the amount of this protein in the nucleus of DN/CL6 cells; the abundance of p65 in the nucleus of untreated DN/CL6 cells was greater than that in the nucleus of untreated SCC-35 cells (FIG. 2A). These data thus show that p65DN interfered with the proteolysis of phosphorylated IκB-α, suggesting that the phosphorylation of this protein is not sufficient for its dissociation from NF-κB. This conclusion is consistent with the previous observation that amino acids 1 to 101 of p65 are required for its binding to IκB-α (36). We therefore propose that p65DN interferes with the inhibitory function of IκB-α by binding to this protein in both the cytoplasm and nucleus.

To determine whether p65DN inhibits the activation of NF-κB, we transfected DN/CL6 cells with the NF-κB-specific luciferase reporter vector pNF-κB-Luc and then examined the effect of IR or TNF-α on luciferase activity. The stimulatory effects of these agents on NF-κB activity were reduced by a factor of 2 to 3 in DN/CL6 cells compared with those apparent in SCC-35 cells (FIG. 2B). Expression of p65DN thus inhibited NF-κB activation.

Effects of p65DN on Cellular Sensitivity to IR

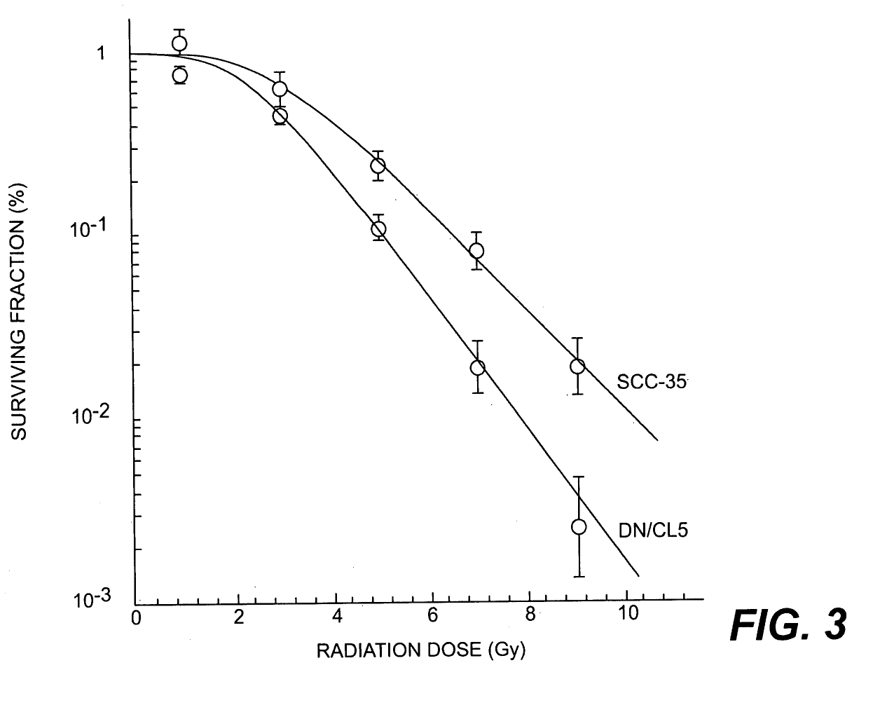

Impaired regulation of NF-κB in certain cell types is associated with intrinsic cellular radiosensitivity (14-16). To examine the role of NF-κB in determining cell sensitivity to radiation, we performed clonogenic survival assays after exposure of SCC-35 and DN/CL6 cells to graded doses of γ-radiation (FIG. 3). The sensitivity of DN/CL6 cells to IR was greater than that of the parental SCC-35 cells. According to the single-hit multitarget model (33), D₀ values were determined to be 1.9 Gy (with α=0.16 and β=0.02) for SCC-35 cells and 1.2 Gy (with α=0.11 and β=0.06) for DN/CL6 cells. Because of the potential pitfalls of clonal heterogeneity associated with determination of radiation sensitivity in a clonal cell population, we also performed survival experiments with pooled transfected clones. We obtained similar results (data not shown), suggesting that the observed p65DN-induced increase in radiation sensitivity is not simply due to clonal selection.

Effects of p65DN on IR-Induced Apoptosis

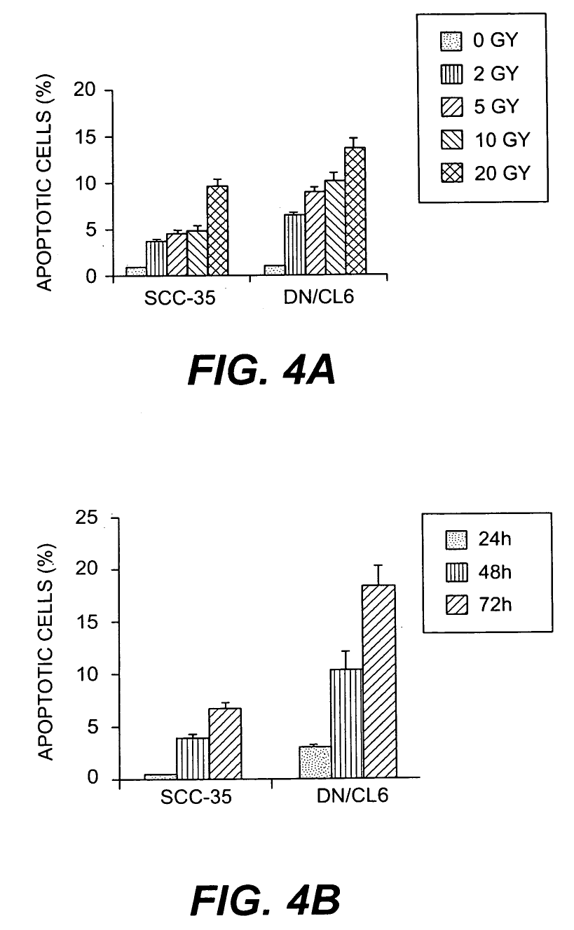

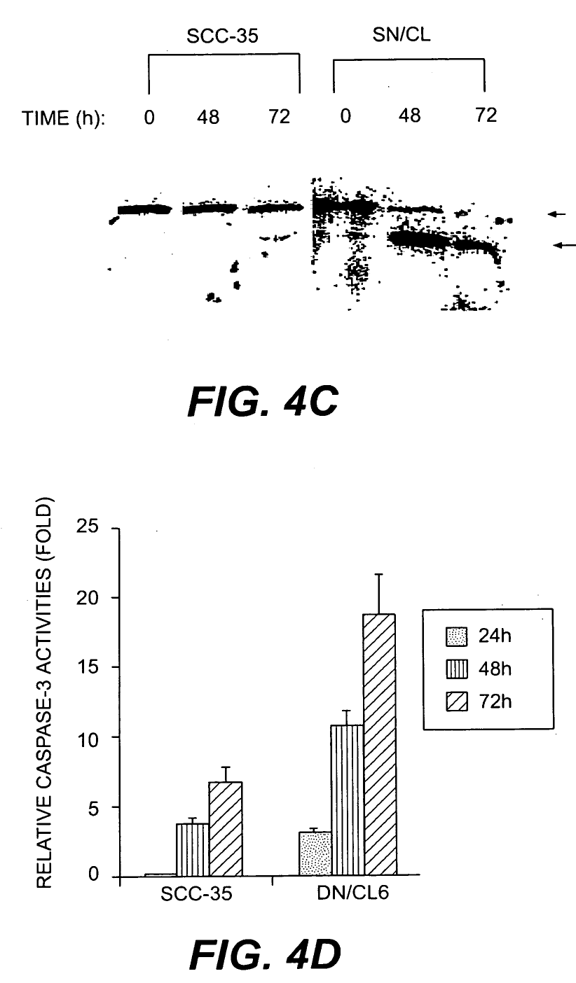

Activation of NF-κB contributes to the protection of many cell types from apoptotic cell death induced by DNA-damaging agents. However, under certain conditions and in certain cell types, activation of NF-κB induces apoptosis (37). Given that clonogenic survival analysis provides a measure of all types of cell death, we next determined the effect of p65DN expression on IR-induced apoptosis. IR induced a dose-dependent increase in the percentage of both SCC-35 and DN/CL6 cells characterized as apoptotic 48 h after irradiation (FIG. 4A). However, whereas the maximum proportion of apoptotic cells was 8% (evident at 20 Gy) for SCC-35 cells, confirming that apoptosis is not a major pathway of cell death in these cells, the maximum apoptotic index was 14% for DN/CL6 cells. The baseline apoptotic index for both cell lines was <1%. The time courses of the changes in the apoptotic index induced by 5 Gy of γ-radiation revealed that a substantial increase in this parameter was apparent at 24 h in DN/CL6 cells but not until 48 h in SCC-35 cells (FIG. 4B).

The nuclear enzyme poly(ADP-ribose) polymerase (PARP), which plays an important role in the repair of DNA damage and in maintenance of genomic integrity (38, 39), is specifically cleaved between its NH₂-terminal DNA-binding domain and its multifunctional COOH-terminal domain by caspase-3 or a caspase-3—like protease early during the execution phase of apoptosis (40). We therefore examined the effects of p65DN on both the cleavage of PARP and caspase-3 activity. Exposure of DN/CL6 cells to γ-radiation at doses as low as 2 Gy resulted in PARP cleavage, whereas PARP cleavage in SCC-35 cells required much larger doses of IR (>20 Gy) (data not shown). Exposure to 5 Gy of γ-radiation induced a substantial increase in PARP cleavage within 48 h in DN/CL6 cells, whereas cleavage of this enzyme remained barely detectable at 72 h in SCC-35 cells (FIG. 4C). Similarly, IR (5 Gy) induced an earlier and more pronounced activation of caspase-3 in DN/CL6 cells than in SCC-35 cells (FIG. 4D). Together, these observations suggest that caspase-3 contributes to the cleavage of PARP during IR-induced apoptosis in SCC-35 cells, that NF-κB protects these cells from apoptosis, and that expression of p65DN potentiates IR-induced apoptosis in the tumor cells.

Discussion

Cellular responses to ionizing radiation (IR) include the activation of signaling pathways that lead to the expression of survival or death factors. In general, activation of NF-κB plays an important role in cellular survival after exposure to stressful stimuli such as IR. Proteins that contribute to radiation-induced NF-κB signaling therefore provide potential targets for sensitization of tumor cells to radiation.

Crystallographic studies have identified the sites of physical interaction between NF-κB (p50-p65) and IκB-α (41-43). The ankyrin repeat 6 and an adjacent partial PEST sequence of IκB-α interact with the NH₂-terminal immunoglobulin-like motif of the Rel homology domain of p65, including residues Tyr²⁰, Glu²², Glu⁴⁹, Arg⁵⁰, His¹⁸¹, Arg²⁴⁶, and Arg²⁹⁴. In addition, ankyrin repeats 1 and 3 of IκB-α interact with sequences encompassing the nuclear localization signal of p65. We have now shown that expression of an NH₂-terminal fragment (residues 1 to 84) of p65 both blocks the degradation of IκB-α after its stimulus-induced phosphorylation and disrupts NF-κB activation, thereby increasing the sensitivity of radiation-resistant human squamous carcinoma cells to IR-induced cell death.

Phosphorylation of IκB proteins and their subsequent degradation is an important mechanism of NF-κB activation. Rapid resynthesis of these proteins results in their entry into the nucleus and removal of NF-κB from its DNA binding sites (41, 44-46). Thus, IκB proteins have been considered potential molecular targets for modulation of NF-κB activation; indeed, such modulation has been achieved by expression of mutant forms of IκB (1). Our data now show that expression of the NH₂-terminal portion of p65 comprising amino acids 1 to 84 results in down-regulation of both the endogenous p65 protein and its mRNA. IκB-α was phosphorylated within 30 min of exposure to IR in both parental SCC-35 cells and cells expressing p65DN. However, whereas subsequent degradation of IκB-α was apparent within 1 h of irradiation in the parental cells, no such degradation was detected for up to 3 h in DN/CL6 cells. These results suggest that the NH₂-terminus of p65 is a potential molecular target for disruption of NF-κB activation through inhibition of IκB-α degradation. They also suggest that the degradation of phosphorylated IκB-α requires an interaction with additional sequences of p65, such as the nuclear localization signal or transcriptional activation domain, or additional events such as a conformation change or phosphorylation of p65.

Disruption of NF-κB activation by expression of IκB-αmutants has been shown to result in an increase in the radiosensitivity of human tumor cells in a cell type-specific manner (37). Our data now show that the radiation-resistant SCC-35 human tumor cells become sensitized to IR-induced cell death as a result of inhibition of NF-κB activation.

Although both necrotic and apoptotic mechanisms of cell death may contribute to antitumor therapy, it is important to understand the role of each in order to tailor treatment to the specific tumor. In most cells, NF-κB mediates cell survival signals, protecting cells from apoptosis. Thus, when NF-κB activation is impaired, as has been achieved by the expression of IκB-α mutants (14, 15, 20), cells undergo apoptosis. A previous study in which NF-κB was targeted by viral gene therapy was performed with cells that were highly sensitive to apoptotic, death (19).

Activation of caspases results in the proteolysis of intracellular proteins that contribute to the maintenance of cellular integrity. PARP is a prominent substrate of caspase-3 during apoptosis. We have now shown that the increase in the sensitivity of SCC-35 cells to IR-induced apoptosis achieved by expression of p65DN was accompanied by an increased extent of PARP cleavage and caspase-3 activation, indicating the importance of caspase-3 as a downstream protease during IR-induced apoptosis. These data also suggest that NF-κB contributes to the regulation of caspase-3 and PARP.

In summary, expression of p65DN resulted in down-regulation of both endogenous p65 protein and its mRNA. It also inhibited proteolysis of phosphorylated IκB-α after cell irradiation suggesting that dissociation and proteolysis of IκB-α require additional events. The binding of p65DN to IκB-α may thus stabilize the phosphorylated form of IκB-α. Disruption of radiation-induced signal transduction through NF-κB represents a means of achieving cellular radiosensitization and provides a basis for new antitumor therapeutic strategies.

### REFERENCES

1. Baldwin A S: The NF-κB and IκB Proteins: New discoveries and insights. Ann Rev Immunol 14:649-681,1996.
2. Beg A A, Baltimore D: An essential role for NF-κB in preventing TNF-a-induced cell death. Science 274:782-784, 1996.
3. Mayo M W, Baldwin A S: The transcription factor NF-κB: control of oncogenesis and cancer therapy resistance. Bioch rt Biophys Acta M55-M62, 2000.
4. Siebenlist U, Franzoso G, Brown K: Structure, regulation and function of NF-κB. Ann Rev Cell Biol 10:405-455,1994.
5. Coleman T A, Kunsch C, Maher M, Ruben S M, Rosen Calif. Acquisition of NFKB1-selective DNA bingding by substitution of four amino acid residues from NFKB1 into RelA. Mol Cell Biol 13: 3850-3859, 1993.
6. Beg A A, Ruben S M, Scheinman R I, Haskill S, Rosen C A, Baldwin A S Jr. Ikappa B interacts with the nuclear localization sequences of the subunits of NF-kappa B: a mechansim for cytoplasmic retention. Genes Dev 6: 1899-18913,1992.
7. DiDonato J A, Mercurio F, Karin M: Phosphorylation of IκB precedes but is not sufficient for its dissociation from NF-κB. Mol Cell Biol 15:1302-1311, 1995.
8. Lin Y-C, Brown K, Siebenlist U. Activation of NF-κB requires proteolysis of the inhibitor IκB: Signal-induced phosphorylation of IκB alone does not release active NF-κB. Proc Natl Acad Sci USA 92:3003-3009, 1995.
9. Didonato J, Mercurio F, Rosette C, Wu-Li J, Suyang H, Ghosh S, Karin M. Mapping of the inducible IκB phosphorylation sites that signal its ubiquitination and degradation. Mol. Cell. Biol. 16:1295-1304, 1996.
10.Brach M A, Hass R, Shwerman M L, Gunji H, Weichselbaum R R, Kufe D W. Ionizing radiation induces expression and binding activity of the nuclear factor kappa B. J. Clin Invest 88:691-695, 1991.
11. Pajonk F, McBride W H. Ionizing radiation affects 26s proteasome function and associated molecular responses, even at low doses. Radiother Oncol. 59:203-12, 2001.
12. Prasad A V, Mohan N, Chandrasekar B, Meltz M L. Activation of nuclear factor kappa B in human lymphoblastoid cells by low-dose ionizing radiation. Radiat Res. 138:367-372,1994.
13. Lee S, Dimtchev A, Larin M, Dritschilo A, Jung M. A Novel Ionizing Radiation-Induced Signaling Pathway that Activates the Transcription factor NF-κB. Oncogene 17:1821-1826, 1998.
14.Jung M, Zhang Y, Dimtchev A, Dritschilo A: Impaired Regulation of Nuclear Factor-κB Results in Apoptosis Induced by Gamma Radiation. Rad Res 149:596-601, 1998.
15.Jung M, Zhang Y, Dritschilo A: Expression of a dominant negative IκB-α modulates hypersensitivity of ataxia telangiectasia fibroblasts to streptonigrin-induced apoptosis. Radiat Oncol Invest 5:265-268, 1997.
16. Pajonk F, Pajonk K, McBride W H. Apoptosis and radiosensitization of hodgkin cells by proteasome inhibition. Int J Radiat Oncol Biol Phys. 47:1025-1032, 2000.
17. Pajouk F. Pajouk K, McBride W H. Inhibition of NF-κB, Clonogenicity, and Radiosensitivity of Human Cancer Cells. J Natl Cancer Inst 91:1956-1960, 1999.
18.Dritschilo A. Radiosensitivity and Transcription Factor NF-κB Inhibition—Progress and Pitfalls (Editorial). J Nat Cancer Inst 22:1910-1911
19.Van Antwerp D J, Martin S J, Kafri T, Green D R, Verma I M: Suppression of TNF-a-induced apoptosis by NF-κB. Science 274:787-789, 1996.
20.Wang C Y, Mayo M W, and Baldwin A S Jr: TNF-and cancer therapy-induced apoptosis: potentiation by inhibition of NF-κB. Science 274:784-787, 1996.
21. Delic J, Masdehors P, Omura S, Cosset J M, Dumont J, Binet J L, Magdelenat H. The proteasome inhibitor lactacystin induces apoptosis and sensitizes chemo- and radioresistant human chronic lymphocytic leukaemia lymphocytes to TNF-alpha-initiated apoptosis. Br J Cancer. 77:1103-1107, 1998.
22. Hallahan D E, Teng M. The proteasome: a molecular target for cancer therapy. Int J Radiat Oncol Biol Phys. 47:859-860, 2000.
23. Russo S M, Tepper J E, Bladwin A S, Liu R, Adams J, Elliott P, Cusack J C: Enhancement of radiosensitivity by proteasome inhibition: implications for a role of NF-κB. Int. J. Radiat Oncol Biol Phys 50:183-193, 2001.
24.Yamagishi N, Miyakoshi J, Takebe H. Enhanced radiosensitivity by inhibition of nuclear factor κB activation in human malignant glioma cells. Int. J. Radiat. Biol 72:157-162, 1997.
25. Teicher B A, Ara G, Herbst R, Palombella V J, Adams J. The proteasome inhibitor PS-341 in cancer therapy. Clin Cancer Res. 5:2638-2645.1999.
26. Frankel A, Man S, Elliott P, Adams J, Kerbel R S. Lack of multicellular drug resistance observed in human ovarian and prostate carcinoma treated with the proteasome inhibitor PS-341. Clin Cancer Res. 6:3719-3728, 2000.
27.Adams J, Palombella V J, Sausville E A, Johnson J, Destree A, Lazarus D D, Maas J, Pien C S, Prakash S, Elliott P J. Proteasome inhibitors: a novel class of potent and effective antitumor agents. Cancer Res. 59:2615-2622, 1999.

28. Sunwoo J B, Chen Z, Dong G, Yeh N, Bancroft C C, Sausville E, Adams J, Elliott P, Van Waes C. Novel proteasome inhibitor PS-341 inhibits activation of nuclear factor-kB, cells survival, tumor growth, and angiogenesis in squamous cell carcinoma. Clinical Cancer Res. 7: 1419-1428, 2001.
29. Harbison M T, Bruns C J, Bold R J et al. Proteosome inhibitor PS-341 effective as an anti-angiogenic agent in the treatment of human pancreatic cancer via the inhibition of NF-kB and subequent inhibition of vascular endothelial growth factor production. Clin Cancer Res5 (Supp): 3812s, #414,1999.
30. Weichselbaum R R, Beckett M A, Schwartz J L, Dritschilo A. Radioresistant Tumor Cells are Present in Head and Neck Carcinomas that Recur after Radiotherapy. Int J Rad One Biol Phys 15:575-579, 1988.
31. Ruben S M, Dillon P J, Schreck R, Henkel T, Chen C H, Maher M, Baeuerle P A, Rosen C A. Isolation of a rel-related human cDNA that potentially encodes the 65-kD subunit of NF-κB. Science 251: 1490-1493, 1991.
32. Jung M, Zhang Y, Lee S A, Dritschilo A. Correction of radiation sensitivity in ataxia telangiectasia cells by a truncated IκB-α. Science 268:1619-1621, 1995.
33. Albright N. Computer programs for the analysis of cellular survival data. Radiat. Res. 112:331-340, 1987.
34.Zhang Y, Dimtchev A, Dritschilo A and Jung M. Ionizing radiation-inducced apoptosis in ataxia telangiectasia fibroblasts: Roles of caspase-9 activation and cellular inhibitor of apoptosis protein-1. Journal of Biological Chemistry 276: 28842-28848, 2001.
35. Kunsch C, Ruben S M, Rosen C A. Selection of optimal kappa B/Rel DNA-binding motifs: interaction of both subunits of NF-kappa B with DNA is required for transcriptional activation. Mol Cell Biol 12: 4412-4421, 1992.
36. Beg A A, Ruben S M, Scheinman R I, Haskill S, Rosen C A, Baldwin A S. IκB interacts with the nuclear localization sequences of the subunits of NF-κB: a mechanism for cytoplasmic retention. Genes & Dev 6: 1899-1913, 1992.
37. Jung M and Dritschilo A. NF-κB signaling pathway as a target for human tumor radiosensitization. Seminars in Radiat. Oncology 11: 346-351, 2001.
38.Thornberry, N. A., Rano, T. A., Peterson, E. P., Rasper, D. M., Timkey, T., Garcia-Calvo, M., Houtzager, V. M., Nordstrom, P. A., Roy, S., Vaillancourt, J. P., Chapman, K. T., Nicholson, D. W. A combinatorial approach defines specificities of members of the caspase family and granzyme B. Functional relationships established for key mediators of apoptosis. J. Biol. Chem. 272:17907-17911.1997.
39. Green, D. and Krocmer, G. The central executioners of apoptosis: caspases or mitochondria? Trans Cell Biol. 8: 267-271, 1998.
40. Nicholson, D. W. and Thornberry, N. A. Caspases: killer proteases. Trends Biochem. Sci. 22: 299-306, 1997.
41. Baeuerle P A. IκB-NF-κB structures: at the interface of inflammation control. Cell 95: 729-731, 1998.
42. Jacobs M D and Harrison S C. Structure of an IκB-α/NF-κB complex. Cell 95: 749-758, 1998.
43. Huxford T, Huang D B, Malek S, Ghosh G. The crystal structure of the IκBα/NF-κB complex reveals mechanisms of NF-κB inactivation. Cell 95: 759-770, 1998.
44.Arenzana-Seisdedos F, Turpin P, Rodriguez M, Thomas D, Hay RT, Virelizer J L, Dargemont C. Nuclear localization of IκB-□promotes active transport of NF-κB from the nucleus to the cytoplasm. J. Cell Sci. 110: 369-378, 1997.
45.Zabel U, and Baeuerle P A. Purified human IκB can rapidly dissociate the complex of the NF-κB transcription factor with its cognate DNA. Cell 61: 255-265, 1990.
46.Zabel U, Henkel T, Silva M S, Baeuerle P A. Nuclear uptake control of NF-κB by MAD-3, an IκB protein present in the nucleus. EMBO J. 12: 201-211, 1993.
47. Beraud C, Henzel W J, Baeuerle P A. Involvement of regulatory and catalytic subunits of phosphoinositide 3-kinase in NF-kappaB activation. Proc Natl Acad Sci USA. 96(2):429-34, 1999 Jan. 19.
48.Tam W F & Sen R. IkappaB family members function by different mechanisms. J. Biol. Chem. 276(11): 7701-4, 2001 Mar. 16.
49. May M J, D'Acquisto F, Madge L A, Glockner J, Pober J S, Ghosh S. Selective inhibition of NF-kappaB activation by a peptide that blocks the interaction of NEMO with the IkappaB kinase complex. Science 289(5484): 1550-4, 2000 Sep. 1.

50. Schottelius A J, Mayo M W, Sartor A B, Baldwin A S Jr. Interleukin-10 signaling blocks inhibitor of kappaB kinase activity and nuclear factor kappa B DNA binding. J. Biol. Chem. 274(45): 31868-74, 1999 Nov. 5.

1. A method of inhibiting or reducing NF-κB activation in a cell comprising:
exposing said cell to a dominant negative NF-κB subunit or fragment thereof such that NF-κB activation is inhibited.

2. The method of claim 1, wherein said dominant negative NF-κB subunit or fragment thereof is a dominant negative p65 subunit or p65 fragment.

3. The method of claim 2, wherein said dominant negative p65 subunit or p65 fragment comprises a DNA binding domain.

4. The method of claim 3, wherein said dominant negative p65 subunit or p65 fragment comprises amino acids ranging from about 1 to about 250 to about 300 of native p65.

5. The method of claim 4, wherein said dominant negative p65 subunit or p65 fragment does not contain an active nuclear localization sequence.

6. The method of claim 4, wherein said dominant negative p65 subunit or p65 fragment does not contain an active transcriptional activation domain.

7. The method of claim 2, wherein said dominant negative p65 subunit or p65 fragment comprises an IκB-alpha binding domain.

8. The method of claim 7, wherein said dominant negative p65 subunit or p65 fragment comprises amino acids 1-101 of native p65.

9. The method of claim 2, wherein said dominant negative p65 subunit or p65 fragment comprises amino acids from native p65 ranging from about 1 to about 100.

10. The method of claim 9, wherein said dominant negative p65 subunit or p65 fragment comprises amino acids from native p65 ranging from about 1 to about 85.

11. The method of claim 10, wherein said dominant negative p65 subunit or p65 fragment comprises amino acids 1-84 from native p65.

12. The method of claim 1, wherein said exposure is mediated by:
transfecting said cell with a DNA encoding said dominant negative NF-κB subunit or fragment thereof, and
expressing the dominant negative NF-κB subunit or fragment thereof encoded by said DNA such that NF-κB activation is inhibited or reduced.

13. The method of claim 1, wherein said exposure is mediated by contacting said cell with said dominant negative NF-κB subunit or fragment thereof coupled to an internalization moiety, wherein said internalization moiety is selected from the group consisting of a peptide internalization sequence, a liposome, and an antibody or an antibody fragment or ligand that binds to a cell surface receptor.

14. The method of claim 13, wherein said cell is contacted with said dominant negative NF-κB subunit or fragment thereof coupled to an internalization moiety in vitro.

15. The method of claim 14, wherein said cell is contacted with said dominant negative NF-κB subunit or fragment thereof coupled to an internalization moiety in vivo.

16. The method of claim 1, wherein said inhibition or reduction of NF-κB activation results in modulation of expression of at least one NF-κB-regulated gene.

17. The method of claim 1 further resulting in a down-regulation of gene expression of native p65.

18. The method of claim 7 wherein IκB-alpha is phosphorylated but not degraded.

19. The method of claim 1, wherein said dominant negative NF-κB subunit or fragment thereof competes with native NF-κB for IκB-alpha binding.

20. The method of claim 1, resulting in increased sensitivity of said cell to ionizing radiation.

21. The method of claim 20, further comprising exposing said cell to ionizing radiation.

22. The method of claim 21, where said cell undergoes radiation-induced apoptosis.

23. The method of claim 22, wherein said cell is a tumor cell.

24. A therapeutic composition comprising a dominant negative NF-κB subunit or fragment thereof coupled to an internalization moiety, wherein said subunit or fragment sensitizes cells to ionizing radiation.

25. The therapeutic composition of claim 24, wherein said dominant negative NF-κB subunit or fragment thereof is a dominant negative p65 subunit or p65 fragment.

26. The therapeutic composition of claim 25, wherein said dominant negative p65 subunit or p65 fragment comprises a DNA binding domain.

27. The therapeutic composition of claim 26, wherein said dominant negative p65 subunit or p65 fragment comprises amino acids ranging from about 1 to about 250 to about 300 of native p65.

28. The therapeutic composition of claim 26, wherein said dominant negative p65 subunit or p65 fragment does not contain an active nuclear localization sequence.

29. The therapeutic composition of claim 26, wherein said dominant negative p65 subunit or p65 fragment does not contain an active transcriptional activation domain.

30. The therapeutic composition of claim 25, wherein said dominant negative p65 subunit or p65 fragment comprises an IκB-alpha binding domain.

31. The therapeutic composition of claim 30, wherein said dominant negative p65 subunit or p65 fragment comprises amino acids 1-101 of native p65.

32. The therapeutic composition of claim 25, wherein said dominant negative p65 subunit or p65 fragment comprises amino acids from native p65 ranging from about 1 to about 100.

33. The therapeutic composition of claim 32, wherein said dominant negative p65 subunit or p65 fragment comprises amino acids from native p65 ranging from about 1 to about 85.

34. The therapeutic composition of claim 33, wherein said dominant negative p65 subunit or p65 fragment comprises amino acids 1-84 from native p65.

35. A therapeutic composition comprising a DNA encoding a dominant negative NF-κB subunit or fragment thereof, wherein said subunit or fragment sensitizes cells to ionizing radiation.

36. The therapeutic composition of claim 35, wherein said dominant negative NF-κB subunit or fragment thereof is a dominant negative p65 subunit or p65 fragment.

37. The therapeutic composition of claim 36, wherein said DNA encoding said dominant negative p65 subunit or p65 fragment encodes a DNA binding domain.

38. The therapeutic composition of claim 37, wherein said DNA encoding said dominant negative p65 subunit or p65 fragment encodes amino acids ranging from about 1 to about 250 to about 300 of native p65.

39. The therapeutic composition of claim 36, wherein said DNA encoding said dominant negative p65 subunit or p65 fragment does not encode a protein that contains an active nuclear localization sequence.

40. The therapeutic composition of claim 39, wherein said DNA encoding said dominant negative p65 subunit or p65 fragment does not encode a protein containing an active transcriptional activation domain.

41. The therapeutic composition of claim 36, wherein said DNA encoding said dominant negative p65 subunit or p65 fragment encodes a protein comprising an IκB-alpha binding domain of native p65.

42. The therapeutic composition of claim 41, wherein said DNA encoding said dominant negative p65 subunit or p65 fragment encodes a protein comprising amino acids 1-101 of native p65.

43. The therapeutic composition of claim 36, wherein said DNA encoding said dominant negative p65 subunit or p65 fragment encodes a protein comprising amino acids from native p65 ranging from about 1 to about 100.

44. The therapeutic composition of claim 43, wherein said DNA encoding said dominant negative p65 subunit or p65 fragment encodes a protein comprising amino acids from native p65 ranging from about 1 to about 85.

45. The therapeutic composition of claim 44, wherein said DNA encoding said dominant negative p65 subunit or p65 fragment encodes a protein comprising amino acids 1-84 from native p65.

46. The therapeutic composition of claim 35, wherein said DNA encoding a dominant negative NF-κB subunit or fragment thereof is operably linked to expression regulatory sequences that permit expression of said DNA in a target cell.

47. The therapeutic composition of claim 46, wherein said target cell is a cancer cell.

48. A method of sensitizing a cell to ionizing radiation comprising contacting said cell with the therapeutic composition of claim 24.

49. The method of claim 48, wherein said cell is a cancer cell.

50. A method of sensitizing a cell to ionizing radiation comprising contacting said cell with the therapeutic composition of claim 46 such that said DNA is taken up and expressed by said cell.

51. The method of claim 50, wherein said cell is a cancer cell.

52. A method of treating cancer, comprising contacting cells of said cancer with a dominant negative NF-κB p65 subunit fragment comprising amino acids from about 1 to about 84 of native p65 coupled to an internalization moiety.

53. The method of claim 52, further comprising exposing said cells to ionizing radiation.

54. A method of treating cancer, comprising contacting cells of said cancer with a DNA encoding a dominant negative NF-κB p65 subunit fragment comprising amino acids from about 1 to about 84 of native p65 such that said DNA is taken up and expressed by said cells.

55. The method of claim 54, wherein said DNA is administered by direct injection at the site of the cancer.

56. The method of claim 54, further comprising exposing said cells to ionizing radiation.

57. A method of treating inflammation in a patient, comprising administering to said patient a therapeutic composition comprising a dominant negative NF-κB p65 subunit fragment comprising amino acids from about 1 to about 84 of native p65 coupled to an internalization moiety.

58. A method of treating inflammation in a patient, comprising administering to said patient a therapeutic composition comprising a DNA encoding a dominant negative NF-κB p65 subunit fragment comprising amino acids from about 1 to about 84 of native p65.

59. The method of claim 58, wherein said DNA is administered by direct injection at the site of said inflammation.

60. The method of claim 57, wherein said inflammation is associated with an inflammatory disease selected from the group consisting of rheumatoid arthritis, ataxia-telangiectasia, asthma, inflammatory bowel disease and atherosclerosis.

61. The method of claim 57 which modulates the expression of a cytokine, immunoreceptor or a gene that responds to oxidative stress.
